(19) United States
(12) Patent Application Publication
(10) Pub. No.: US 2025/0000641 A1
(43) Pub. Date: Jan. 2, 2025

(54) REINFORCEMENT COMPONENT FOR HIGH-MYOPIA POSTERIOR SCLERA

(71) Applicant: GUANGZHOU VESBER BIOTECHNOLOGY CO., LTD., Guangzhou (CN)

(72) Inventor: Qianying GAO, Guangzhou (CN)

(73) Assignee: GUANGZHOU VESBER BIOTECHNOLOGY CO., LTD., Guangzhou, GD (CN)

(21) Appl. No.: 18/882,776

(22) Filed: Sep. 12, 2024

Related U.S. Application Data
(63) Continuation of application No. PCT/CN2023/079653, filed on Mar. 3, 2023.

(30) Foreign Application Priority Data
Sep. 22, 2022 (CN) 202211159068.5

(51) Int. Cl. A61F 2/14

(52) U.S. Cl. CPC A61F 2/15; A61F 2230/0008

(57) ABSTRACT

A reinforcement component for a high-myopia posterior sclera, including a support pad and support strips connected to the support pad. The support pad is provided with an avoidance portion at an edge thereof and through-holes for blood vessels to pass through. Each through-hole is in a form of an ellipse with a long axis parallel with an edge of the avoidance portion and a short axis. The avoidance portion and the through-holes are provided such that even if the size of the through-hole is compressed by the avoidance portion, the through-hole is elliptical with its long axis set along the edge of the avoidance portion, the size of the through-hole can be expanded. The ratio of the long axis to the short axis of the through-hole is not less than 8/7, and not more than 20/9, which facilitates the blood vessels to pass through the through-hole.

### TECHNICAL FIELD

The present disclosure relates to the field of medical devices, and in particular, to a reinforcement component for a high-myopia posterior sclera.

### BACKGROUND

In the field of medical ophthalmology, a reinforcement component for a high-myopia posterior sclera usually consists of a support pad and three support strips, and a titanium plate is encapsulated in the support pad, and the support pad is provided with through holes for blood vessels to pass through. The reinforcement component for a high-myopia posterior sclera is directed to the treatment of high myopia as well as high myopia accompanied by posterior scleral staphyloma, retinal cleavage, laceration or detachment, and is used to shorten the length of the eye axis in the macular region and to reduce the refractive error, and can play a role in adjusting the eye axis of the eye. The reinforcement component for a high-myopia posterior sclera needs to be implanted into the eye when use. The support pad and the support strips are adopted for fixing the posterior sclera for a long period of time to enable the cleavage and the visual acuity to be improved.

After the reinforcement component for a high-myopia posterior sclera is implanted into the eye to fix the posterior sclera, the support pad is prone to squeeze and damage the optic nerve. In order to avoid damage to the optic nerve, the current reinforcement component for a high-myopia posterior sclera is usually provided with an avoidance portion at the edge of the support pad, and the avoidance portion can avoid damage to the optic nerve. However, providing an avoidance portion at the edge of the support pad can reduce the size of the support pad, resulting in the size of the through-holes adjacent to the avoidance portion being compressed, which is not favorable to the passage of blood vessels.

### SUMMARY

There is a desire to provide an improved reinforcement component for a high-myopia posterior sclera.

In one aspect, the present invention provides a reinforcement component for a posterior sclera, comprising a support pad and a plurality of support strips connected to the support pad, the support pad being provided with an avoidance portion at an edge thereof, and the support pad defining through-holes for blood vessels to pass through adjacent to the avoidance portion. Each of the through-holes is in a form of an ellipse; each ellipse through-hole comprises a long axis parallel to an edge of the avoidance portion and a short axis perpendicular to the long axis; a ratio of the long axis to the short axis of each ellipse through-hole is not less than 8/7 and not greater than 20/9.

Preferably, the plurality of support strips comprises a first support strip, a second support strip and a third support strip each of which is provided with a graduated scale.

Preferably, the long axis of the ellipse through-hole is not less than 1.6 mm and not greater than 2.0 mm; and the short axis of the ellipse through-hole is not less than 0.9 mm and not greater than 1.4 mm.

Preferably, two said through-holes are provided, and the two said through-holes are aligned along the long axis.

Preferably, a shortest distance between the two through-holes is not less than 1.35 mm.

Preferably, a shortest distance between the through holes and the edge of the avoidance portion is not less than 1.14 mm.

Preferably, a surface of each support strip is provided with a plurality of grooves configured for contacting with eyeballs for anti-sliding, and an edge of each groove is provided with a chamfer.

Preferably, an end of the support strip facing outwardly is rounded.

Preferably, the support pad comprises a surface which is inwardly recessed to form a concaved structure, and a surface of the concaved structure of the support pad is rounded and smooth.

The present disclosure has the following beneficial effects: the edge of the support pad is provided with an avoidance portion, and the through-holes for blood vessel passage are provided on the support pad adjacent to the avoidance portion, such that even if the avoidance portion compresses/limits the sizes of the through-holes in one direction, each through-hole is elliptical in shape and its long axis is set along the edge of the avoidance portion, so as to expand the size of the through-hole in another direction, and the ratio of the long axis of the through-hole to the short axis of the through-hole is not less than 8/7 and not more than 20/9, such that the through-hole is elliptical and not excessively flattened, which is favorable for the passage of the blood vessel.

### BRIEF DESCRIPTION OF DRAWINGS

In order to more clearly illustrate the technical solutions in the embodiments of the present disclosure, the following will briefly introduce figures that need to be used in the description of the embodiments. Obviously, the figures in the following description are only some of the embodiments of the present disclosure, and other figures may be obtained from these figures without inventive efforts for those skilled in the art.

For a more complete understanding of the present disclosure and its beneficial effects, the following description will be given in conjunction with the figures. Among them, the same reference characters in the following description denote the same parts.

FIG. 1 is a perspective view of a reinforcement component for a high-myopia posterior sclera provided in an embodiment according to the present disclosure;

FIG. 2 is a front view of the reinforcement component for a high-myopia posterior sclera provided in an embodiment according to the present disclosure;

FIG. 2a is a rear view of the reinforcement component for a high-myopia posterior sclera provided in an embodiment according to the present disclosure; and

FIG. 3 is a side view of the reinforcement component for a high-myopia posterior sclera provided in an embodiment according to the present disclosure.

### DESCRIPTION OF THE REFERENCE CHARACTERS

10, reinforcement structure for a high-myopia posterior sclera; 100, support pad; 101, positioning hole; 102, through-hole; 103, central hole; 104, encircling hole; 105, avoidance portion; 200, first support strip; 201, first groove; 300, second support strip; 301, second groove; 400, third support strip; 401, third groove; 500, reinforcement member.

### DESCRIPTION OF EMBODIMENTS

The inventiveness according to the present disclosure is described in further detail below in connection with specific embodiments.

Embodiments of the present disclosure provide a reinforcement component for a high-myopia posterior sclera to solve the problems of existing posterior scleral reinforcement devices that have the problem of giving the patient a heavy foreign body sensation after implantation, the risk of damaging the eyes, the problem of the patient's foreign body sensation, the problem of not being easy for the surgeon to adjust the device during surgery, and the problem of being prone to the risk of damaging the optic nerve. The following will be described in conjunction with the figures.

The reinforcement component for a high-myopia posterior sclera provided by embodiments of the present disclosure is exemplary. In the present application, high myopia is defined as a SER (spherical equivalent refractive error)≤−6.0 D.

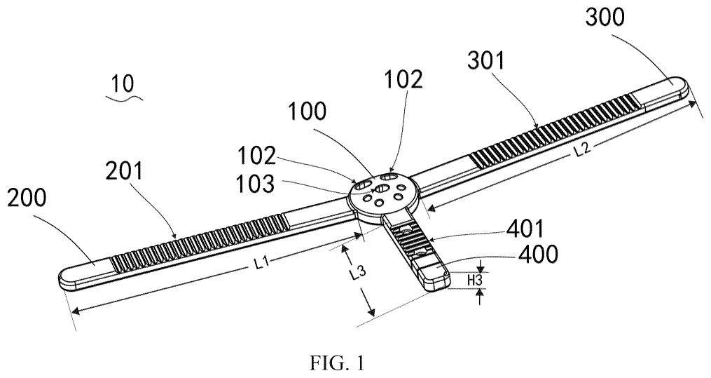

FIG. 1 illustrates a reinforcement component for a high-myopia posterior sclera provided by embodiments of the present disclosure.

In order to more clearly illustrate the structure of the reinforcement component for a high-myopia posterior sclera, the reinforcement component for a high-myopia posterior sclera will be described below in conjunction with the figures.

Exemplarily, referring to FIG. 1, the reinforcement component for a high-myopia posterior sclera includes a support pad 100, a first support strip 200, a second support strip 300 and a third support strip 400. The first support strip 200, the second support strip 300 and the third support strip 400 all are connected to the support pad 100. The first support strip 200, the second support strip 300 and the third support strip 400 are rounded at outwardly facing ends thereof. The ends of the first support strip 200 and the second support strip 300 have smooth chamfered rounded ends, which facilitates the implantation of the reinforcement component into the eyes by a healthcare provider, and also reduces the feeling of a foreign body after implantation. The third support strip 400 is capable of bending and the bending arc can be adjusted according to the condition of the eyes.

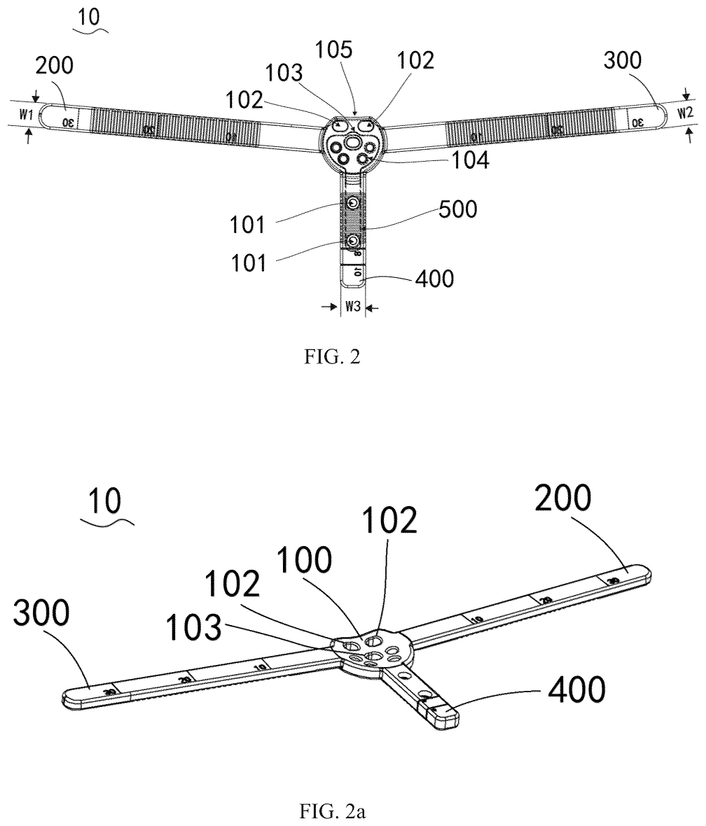

In some of the embodiments, each of the first support strip 200, the second support strip 300 and the third support strip 400 is provided with a scale, as shown in FIG. 2. FIG. 2 shows the reinforcement component for a high-myopia posterior sclera after being flipped over, and thus the numbers of the scale on FIG. 2 are in a state of being flipped over.

The above-described reinforcement component for a high-myopia posterior sclera can be adopted for treatment of high myopia and high myopia accompanied by posterior scleral staphyloma, retinal cleavage, laceration or detachment, and configured for shortening the length of the eye axis of the macula and reducing the refractive error, such that it can play a role in adjusting the eye axis of the eyeball, and it can fixate the posterior sclera for a long period of time to make the cleavage and the visual acuity improved.

In some embodiments, the first support strip 200 has graduated scales on it from an end proximate to the support pad 100 to an end remote from the support pad 100. The second support strip 300 has graduated scales on it from an end proximate to the support pad 100 to an end remote from the support pad 100.

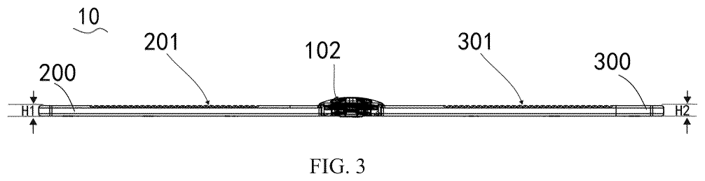

In some of the embodiments, as shown in FIG. 3, the thickness H1 of the first support strip 200 is 0.5-2.5 mm. e.g., in one example, the thickness H1 of the first support strip 200 is 0.5 mm, and in another example, the thickness H1 of the first support strip 200 is 2.5 mm. It is understandable that, in other examples, the thickness H1 of the first support strip 200 may also be 0.6 mm, 0.7 mm, 1.0 mm, 1.2 mm, 1.5 mm, 1.8 mm, 1.9 mm, 2.0 mm, 2.1 mm, 2.3 mm, 2.4 mm, or other parameters.

In some embodiments, as shown in FIG. 2, the width W1 of the first support strip 200 is 1.0-4.0 mm. For example, in one specific example, the width W1 of the first support strip 200 is 1.0 mm, and in another specific example, the width W1 of the first support strip 200 is 4.0 mm. It is understandable that, in other examples, the width W1 of the first support strip 200 may also be 1.5 mm, 2 mm, 2.5 mm, 3 mm, 3.5 mm, or other parameters.

In some embodiments, as shown in FIG. 1, the length L1 of the first support strip 200 is 15-50 mm. For example, in one of the specific examples, the length L1 of the first support strip 200 is 15 mm; in another specific example, the length L1 of the first support strip 200 is 50 mm. It is understandable that, in the other examples, the length L1 of the first support strip 200 may also be 17 mm, 20 mm, 23 mm, 25 mm, 28 mm, 30 mm, 32 mm, 35 mm, 37 mm, 40 mm, 42 mm, 44 mm, 46 mm, 48 mm, 49 mm, or other parameters.

In some embodiments, as shown in FIG. 3, the thickness H2 of the second support strip 300 is 0.5-2.5 mm. For example, in one specific example, the thickness H2 of the second support strip 300 is 0.5 mm, and in another specific example, the thickness H2 of the second support strip 300 is 2.5 mm. It is not difficult to understand that, in other examples, the thickness H2 of the second support strip 300 may also be 0.6 mm, 0.7 mm, 1.0 mm, 1.2 mm, 1.5 mm, 1.8 mm, 1.9 mm, 2.0 mm, 2.1 mm, 2.3 mm, 2.4 mm, or other parameters.

In some embodiments, as shown in FIG. 2, the width W2 of the second support strip 300 is 1.0-4.0 mm. For example, in one specific example, the width W2 of the second support strip 300 is 1.0 mm, and in another specific example, the width W2 of the second support strip 300 is 4.0 mm. It is understandable that, in other examples, the width W2 of the second support strip 300 may also be 1.5 mm, 2 mm, 2.5 mm, 3 mm, 3.5 mm or other parameters.

In some embodiments, as shown in FIG. 1, the length L2 of the second support strip 300 is 15-50 mm. For example, in one specific example, the length L2 of the second support strip 300 is 15 mm; and in another specific example, the length L2 of the second support strip 300 is 50 mm. It is understandable that, in the other examples, the length L2 of the second support strip 300 may also be 17 mm, 20 mm, 23 mm, 25 mm, 28 mm, 30 mm, 32 mm, 35 mm, 37 mm, 40 mm, 42 mm, 44 mm, 46 mm, 48 mm, 49 mm, or other parameters.

In some embodiments, as shown in FIG. 1, the thickness H3 of the third support strip 400 is 0.5-2.5 mm. For example, in one specific example, the thickness H3 of the third support strip 400 is 0.5 mm, and in another specific example, the thickness H3 of the third support strip 400 is 2.5 mm. It is understandable that, in other examples, the thickness H3 of the third support strip 400 may also be 0.6 mm, 0.7 mm, 1.0 mm, 1.2 mm, 1.5 mm, 1.8 mm, 1.9 mm, 2.0 mm, 2.1 mm, 2.3 mm, 2.4 mm, or other parameters.

In some embodiments, as seen in FIG. 2, the width W3 of the third support strip 400 is 1.0-5.0 mm. For example, in one specific example, the width W3 of the third support strip 400 is 1.0 mm, and in another specific example, the width W3 of the third support strip 400 is 5.0 mm. It is not difficult to understand that, in other examples, the width W3 of the third support strip 400 may also be 1.5 mm, 2 mm, 2.5 mm, 3 mm, 3.5 mm, 4 mm, 4.5 mm, or other parameters.

In some of these embodiments, as shown in FIG. 1, the length L3 of the third support strip 400 is 5-25 mm. For example, in one specific example, the length L3 of the third support strip 400 is 5 mm; and in another specific example, the length L3 of the third support strip 400 is 25 mm. It is not difficult to understand that, in the other examples, the length L3 of the third support strip 400 may also be 7 mm, 8 mm, 10 mm, 12 mm, 15 mm, 17 mm, 19 mm, 20 mm, 21 mm, 22 mm, 24 mm, or other parameters.

In some embodiments, the reinforcement component for a high-myopia posterior sclera further comprises a reinforcement member 500. The reinforcement member 500 is attached to the support pad 100 for reinforcing the support pad 100.

In some embodiments, the reinforcement member 500 is a titanium sheet.

In some embodiments, the third support strip 400 defines a plurality of positioning holes 101, and the reinforcement member 500 is connected to the support pad 100 through the positioning holes 101.

In some embodiments, the edge of the support pad 100 has a chamfer, which is so provided that after the reinforcement component for a high-myopia posterior sclera is implanted into the human eye, the optic nerve will not be damaged even if the reinforcement member 500, e.g. a titanium sheet, moves.

The position of the reinforcement member 500 within the support pad 100 is improved such that the edge of the reinforcement member 500 is completely located within the edge of the support pad 100, i.e., the edge of the reinforcement member 500 does not protrude beyond the edge of the support pad 100, which prevents the reinforcement member 500, such as the titanium sheet, from protruding and cutting the optic nerve. In addition, the reinforcement member 500 can increase the amount of top pressure on the product to effectively improve or reset the area where the cleavage has occurred.

Referring to FIG. 2a, in some embodiments, the surface of the support pad 100 is inwardly recessed to form a concaved structure, and the surface of the concave structure of the support pad 100 is rounded. The concaved structure of the support pad 100 may be designed by a computer to finely simulate the eye axis eyeball.

In some embodiments, the thickness of the support pad 100 is 1.0-4.0 mm, which is designed to make the reinforcement component lighter and thinner as a whole, and can reduce the foreign body sensation of the patient and increase the comfort during use. For example, in one specific example, the thickness of the support pad 100 is 1.0 mm, and in another specific example, the thickness of the support pad 100 is 4.0 mm. It is not difficult to understand that, in other examples, the thickness of the support pad 100 may also be 1.5 mm, 2 mm, 2.5 mm, 3 mm, 3.5 mm, or other parameters.

In some embodiments, the surface of the first support strip 200 defines a plurality of first grooves 201. The first grooves 201 are for contacting with the eyeball to increase the friction between the first support strip 200 and the eyeball, to thereby prevent the reinforcement component from sliding within the eye. Preferably, the edge of each first groove 201 is provided with a chamfer. The first grooves 201 facilitate ligation and fixation of the reinforcement component for a high-myopia posterior sclera with the eye, reducing the risk of the reinforcement component detaching from the eyeball. The first grooves 201 with the chamfers facilitate to reduce foreign body sensation when they contact with the eye, such that it is safer.

In some embodiments, the surface of the second support strip 300 defines a plurality of second grooves 301. The second grooves 301 are for contacting with the eyeball to increase the friction between the second support strips 300 and the eyeball, to prevent the reinforcement component for a high-myopia posterior sclera from sliding within the eye. Preferably, the edge of each second groove 301 is provided with a chamfer. The second grooves 301 facilitate ligation and fixation of the reinforcement component for a high-myopia posterior sclera, reducing the risk of the reinforcement component detaching from the eyeball. The second grooves 301 with the chamfers facilitate to reduce foreign body sensation when they contact with the eye, such that it is safer.

In some of these embodiments, the surface of the third support strip 400 has a plurality of third grooves 401. The third grooves 401 are in contact with the eye to increase the friction between the third support strips 400 and the eye, to prevent the reinforcement component for a high-myopia posterior sclera from sliding within the eye. Preferably, the edge of each third groove 401 is provided with a chamfer. The third grooves 401 facilitate ligation and fixation of the reinforcement component for a high-myopia posterior sclera, avoiding the risk of dislodging the reinforcement component for a high-myopia posterior sclera, and at the same time the third groove 401 with the chamfer produces less foreign body sensation in contact with the eye, such that it is safer.

In some embodiments, the angle between the first support strip 200 and the second support strip 300 is 120°-185°. For example, in one embodiment, the angle between the first support strip 200 and the second support strip 300 adjacent thereto is 120°. In another embodiment, the angle between the first support strip 200 and the second support strip 300 adjacent thereto is 185°. Preferably, in one embodiment, the angle formed between the adjacent first support strip 200 and second support strip 300 is 170°.

In some embodiments, the edge of the support pad 100 is provided with an avoidance portion 105, and the avoidance portion 105 can avoid damaging the optic nerve. The avoidance portion 105 may be located at a position between the first support strip 200 and the second support strip 300 and opposite from the third support strip 400.

In some embodiments, two ellipse through-holes 102 are defined in the support pad 100 adjacent to the avoidance portion 105. A long axis of each ellipse through-hole 102 is parallel to an edge of the avoidance portion 105, and the two ellipse through-holes 102 are arranged along the edge of the avoidance portion 105 at an interval. The ellipse through-holes 102 allow blood vessels to pass through these holes. In some embodiments, the long axis of the through-hole 102 is 1.6-2.0 mm. For example, in one specific example, the long axis of the through-hole 102 is 1.6 mm, and in another specific example, the long axis of the through-hole 102 is 2.0 mm. It is understandable that, in other example, the long axis of the through-hole 102 may also be 1.7 mm, 1.8 mm, 1.9 mm, or other parameters.

In some embodiments, the short axis of the through-hole 102 is 0.9-1.4 mm. For example, in one specific example, the short axis of the through-hole 102 is 0.9 mm, and in another specific example, the short axis of the through-hole 102 is 1.4 mm. It is understandable that, in other examples, the short axis of the through-hole 102 may also be 1.0 mm, 1.1 mm, 1.2 mm, 1.3 mm, or other parameters.

Based on the dimensions of the long axis and the short axis of the through-hole 102, it can be calculated that the ratio of the long axis to the short axis of the through-hole 102 has a minimum value of 1.6 mm/1.4 mm=8/7 and a maximum value of 2.0 mm/0.9 mm=20/9, i.e., the ratio of the long axis to the short axis of the through-hole 102 is not less than 8/7 and not greater than 20/9.

In some embodiments, the shortest distance between the two through-holes 102 is not less than 1.35 mm, such that the part between the two through-holes 102 is not too thin, resulting in that the support pad 100 has a certain strength. For example, in one specific example, the shortest distance between the two through-holes 102 is 1.35 mm, and in another specific example, the shortest distance between the two through-holes 102 is 1.40 mm. It is understandable that, in other examples, the shortest distance between the two through-holes 102 may also be 1.38 mm, 1.42 mm, 1.45 mm, or other parameters.

In some embodiments, the shortest distance between the through holes 102 and the edge of the avoidance portion 105 is not less than 1.14 mm, such that the part between the through holes 102 and the edge of the avoidance portion 105 is not too thin, resulting in that the support pad 100 has a certain strength. For example, in one specific example, the shortest distance between the through-hole 102 and the edge of the avoidance portion 105 is 1.14 mm; and in another specific example, the shortest distance between the through-hole 102 and the edge of the avoidance portion 105 is 1.18 mm. It is understandable that, in other examples, the shortest distance between the through-hole 102 and the edge of the avoidance portion 105 may be 1.20 mm, 1.22 mm, 1.25 mm, or other parameters.

In some embodiments, the support pad 100 is provided with a central hole 103. The central hole 103 allows blood vessels to grow out through the hole.

In some embodiments, the support pad 100 is provided with a plurality of encircling holes 104 surrounding the central hole 103. The encircling holes 104 allow blood vessels to grow out through these holes.

In the above-mentioned reinforcement component for a high-myopia posterior sclera, when in use, the support pad 100 is implanted onto the sclera corresponding to the macular portion of the eye, the bending curvature of the third support strip 400 can be adjusted according to the condition of the eye. The rounded and smooth-like structures of the ends of the first support strip 200 and the second support strip 300 can facilitate the doctor to quick extend the first support strip 200 and the second support strip 300 through the external rectus muscle, the inferior rectus muscle and the inferior oblique muscle.

The above-mentioned reinforcement component for a high-myopia posterior sclera has the following beneficial effects relative to the conventional reinforcement device for a posterior sclera:
(1) The ends of the first support strip 200, the second support strip 300 and the third support strip 400 of the above-mentioned reinforcement component for a high-myopia posterior sclera are rounded and smooth, such that the risk of injury to the eyeball thereof can be avoided, and the patient's sense of foreign body can be reduced.
(2) The support pad 100 of the above-mentioned reinforcement component for a high-myopia posterior sclera has been designed to be a concaved structure that can wrap around the eye axis eyeball after fine computer simulation of the eye axis eyeball, such that it can be more compatible with the eye axis eyeball, and the effect of top pressure is better.
(3) The first support strip 200, the second support strip 300 and the third support strip 400 of the above-mentioned reinforcement component for a high-myopia posterior sclera are all designed with a clear graduated scale, such that the medical personnel can be allowed to better judge the implantation position during the surgery.
(4) The first support strip 200, the second support strip 300 and the third support strip 400 of the above-mentioned reinforcement component for a high-myopia posterior sclera are all provided with corresponding grooves with chamfers, such as the first grooves 201, the second grooves 301, and the third grooves 401, such that the reinforcement component for a high-myopia posterior sclera can be prevented from slipping inside the eye, and it is safer.
(5) The ellipse through holes 102 on the support pad 100 of the above-mentioned reinforcement component for a high-myopia posterior sclera can allow blood vessels to grow out better.

As described above is only an embodiment of the present disclosure, and does not limit the claim scope of patent. Non-substantial changes or substitutions made by a person skilled in the art on the basis of the present disclosure still fall within the claim scope of patent.

What is claimed is:

1. A reinforcement component for a high-myopia posterior sclera, comprising a support pad and a plurality of support strips connected to the support pad, the support pad being provided with an avoidance portion at an edge thereof, and the support pad defining through-holes for blood vessels to pass through adjacent to the avoidance portion;
wherein each of the through-holes is in a form of an ellipse;
each ellipse through-hole comprises along axis parallel to an edge of the avoidance portion and a short axis perpendicular to the long axis;
a ratio of the long axis to the short axis of each ellipse through-hole is not less than 8/7 and not greater than 20/9; and
the plurality of support strips comprises a first support strip, a second support strip and a third support strip each of which is provided with a graduated scale.

2. The reinforcement component for a high-myopia posterior sclera according to claim 1, wherein the long axis of the ellipse through-hole is not less than 1.6 mm and not greater than 2.0 mm; and the short axis of the ellipse through-hole is not less than 0.9 mm and not greater than 1.4 mm.

3. The reinforcement component for a high-myopia posterior sclera according to claim 1, wherein two said through-holes are provided, and the two said through-holes are aligned along the long axis.

4. The reinforcement component for a high-myopia posterior sclera according to claim 2, wherein a shortest distance between the two through-holes is not less than 1.35 mm.

5. The reinforcement component for a high-myopia posterior sclera according to claim 1, wherein a shortest distance between the through holes and the edge of the avoidance portion is not less than 1.14 mm.

6. The reinforcement component for a high-myopia posterior sclera according to claim 1, wherein a surface of each support strip is provided with a plurality of grooves configured for contacting with eyeballs for anti-sliding, and an edge of each groove is provided with a chamfer.

7. The reinforcement component for a high-myopia posterior sclera according to claim 1, wherein an end of the support strip facing outwardly is rounded.

8. The reinforcement component for a high-myopia posterior sclera according to claim 1, wherein the support pad comprises a surface which is inwardly recessed to form a concaved structure, and a surface of the concaved structure of the support pad is rounded and smooth.
